(19) United States
(12) Patent Application Publication
(10) Pub. No.: US 2013/0004982 A1
(43) Pub. Date: Jan. 3, 2013

(54) METHOD AND APPARATUS FOR MAGNETIC FLOW CYTOMETRY

(75) Inventors: Ludwig Bär, Erlangen (DE); Oliver Hayden, Herzogenaurach (DE); Michael Johannes Helou, Regensburg (DE); Mischa Megens, Millbrae, CA (US); Mathias Reisbeck, Obertraubling (DE); Manfred Rührig, Lauf a.d. Pegnitz (DE); Sandro Francesco Tedde, Erlangen (DE)

(73) Assignees: The Regents of the University of California, Berkeley, CA (US); Siemens Aktiengesellschaft, Munich (DE)

(21) Appl. No.: 13/172,070

(22) Filed: Jun. 29, 2011

(51) Int. Cl. C12Q 1/02; C12M 1/42; B82Y 15/00

(52) U.S. Cl. 435/29; 435/287.1; 977/838; 977/902

(57) ABSTRACT

A magnetic flow cytometry apparatus for detection of cells labeled with magnetic nanoparticles has at least one pair of oppositely oriented magnets to provide between the magnets a first magnetic field region with a low magnetic field strength and to provide at poles of the magnets second magnetic field regions with a high magnetic field strength. The magnetic labeled cells provided within a flow input into the magnetic flow cytometry apparatus are enriched in at least one of the second magnetic field regions and supplied to the first magnetic field region, where a magnetic field is applied to the enriched magnetic labeled cells to measure the magnetic relaxation of the magnetic labeled cells in response to the applied magnetic field.

### BACKGROUND

Described below are a method and an apparatus for particle sorting and more particularly a method and an apparatus for magnetic flow cytometry for detection of cells labeled with magnetic nanoparticles.

Flow cytometry is a known technique for counting and examining microscopic particles such as cells and chromosomes by suspending them in a stream of fluid and supplying them to a detection apparatus. In a known fluorescence based cytometry apparatus a beam of laser light having a single wavelength is directed onto a hydro dynamically focused stream of fluid comprising the microscopic particles to be detected. Fluorescent chemicals attached to the particles can be excited into emitting light. The fluorescent light can be picked up by detectors to derive various types of information about the physical and chemical structure of the particles within the fluid. Fluorescence based systems allow a multiplexed readout wherein several fluorescence colors can be simultaneously recorded from multi labeled cells in high-end optical fluorescence based cytometry apparatuses. However, a fluorescence based flow cytometry apparatus has the disadvantage that the effort for sample preparation such as hemolysis and fluorescence labelling is high and cumbersome.

### SUMMARY

Accordingly, an aspect is to provide a method and a cytometry apparatus that allows an efficient detection of cells within a fluid with easy sample preparation.

Described below is a magnetic flow cytometry apparatus for detection of cells labeled with magnetic nanoparticles, including at least one pair of oppositely oriented magnets to provide between the magnets a first magnetic field region with a low magnetic field strength and to provide at poles of the magnets second magnetic field regions with a high magnetic field strength, wherein the magnetic labeled cells provided within a flow input into the magnetic flow cytometry apparatus are enriched in at least one of the magnetic field regions and supplied to the first magnetic field region, where a magnetic field is applied to the enriched magnetic labeled cells to measure the magnetic relaxation of the magnetic labeled cells in response to the applied magnetic field.

In a possible embodiment the applied magnetic field is a pulsed magnetic field and the magnetic relaxation is measured by observing a decay time of the magnetic relaxation in the time domain.

In an alternative embodiment the applied magnetic field is modulated sinusoidally and a phase of the magnetization signal is measured to measure the magnetic relaxation in the frequency domain.

This embodiment has the advantage that it is generally easier to drive an electromagnet harmonically than to pulse it.

The magnetic flow cytometry apparatus does not require a sophisticated sample preparation and, consequently, the time to answer of tests, in particular clinical tests, is reduced considerably in comparison to a known flow cytometry apparatus. The magnetic flow cytometry apparatus allows that sample enrichment is conveniently integrated with cell detection. The resulting speed-up and improved ease of use is a considerable advantage in practical applications.

In a possible embodiment of the magnetic flow cytometry apparatus, the magnetic labeled cells are supplied into the magnetic cytometry apparatus through at least one micro-fluidic channel in a laminar flow.

In a further possible embodiment of the magnetic flow cytometry apparatus, at least one magnetic sensor is provided within the first magnetic field region and adapted to measure the magnetic relaxation of the magnetic labeled cells.

Accordingly, it is possible to distinguish cells which have been labeled with different super paramagnetic markers and having different decay times in their magnetization properties.

In a possible embodiment of the magnetic flow cytometry apparatus, at least one excitation coil or excitation wire is located close to the first magnetic field region and is adapted to generate the magnetic field applied to the enriched magnetic labeled cells.

In a possible embodiment of the magnetic flow cytometry apparatus, the at least one pair of oppositely oriented magnets may be permanent magnets.

In an alternative embodiment of the magnetic flow cytometry apparatus, the at least one pair of oppositely oriented magnets may be electrical coils or wires.

In a possible embodiment of the magnetic flow cytometry apparatus, the first magnetic field region may have a low magnetic field strength of less than 1 m Tesla.

In a further possible embodiment of the magnetic flow cytometry apparatus, the second magnetic field region may have a high magnetic field strength of more than 10 m Tesla.

In a possible embodiment of the magnetic flow cytometry apparatus, the magnetic field is applied to the enriched magnetic labeled cells continuously.

In an alternative possible embodiment of the magnetic flow cytometry apparatus the magnetic field is applied to the enriched magnetic labeled cells discontinuously.

In a possible embodiment of the magnetic flow cytometry apparatus a cell counter is provided which counts the number of magnetic labeled cells having the same measured magnetic relaxation time in response to the applied magnetic field.

In a possible embodiment of the magnetic flow cytometry apparatus, the magnetic labeled cells enriched in a second magnetic field region are aligned by ferro-magnetic lines provided on the inner surface of the micro-fluidic channel to pass the magnetic sensor closely.

In a possible embodiment of the magnetic flow cytometry apparatus, the excitation pulse length of a pulse of the pulsed magnetic field applied to the enriched and aligned magnetic labeled cells is shorter than a passage time of the magnetic labeled cells when passing the magnetic sensor.

In a possible embodiment of the magnetic flow cytometry apparatus, the magnetic sensor includes magnetic sensor elements connected to a measurement bridge circuit.

In a possible embodiment of the magnetic flow cytometry apparatus, the magnetic sensor elements may be giant magnetoresistance (GMR), tunnel magnetoresistance (TMR) or anisotropic magnetoresistance (AMR) sensor elements.

A method for detection of cells labeled with magnetic nanoparticles may include:
enriching magnetic labeled cells of an input flow in a magnetic field region having a high magnetic field strength,
applying a magnetic field to the enriched magnetic labeled cells in a magnetic field region having a low magnetic field strength and
measuring the magnetic relaxation of the magnetic labeled cells in response to the applied magnetic field.

In a possible embodiment of the magnetic flow cytometry apparatus, the enriched magnetic labeled cells are aligned by ferro-magnetic lines provided on an inner surface of a micro-fluidic channel adapted to supply the input flow of enriched magnetic cells to the magnetic field region having a low magnetic field strength.

In a possible embodiment of the method, the magnetic field regions are generated by at least one pair of oppositely oriented magnets.

In a still further embodiment of the method, the magnetic labeled cells are supplied as an input flow from a fluid container holding a fluid containing magnetic labeled cells.

In a further possible embodiment of the method, cells are labeled by adding one or several different markers each including magnetic nanoparticles to the fluid container wherein the markers attach themselves to specific receptors on the surface of the cells.

In a still further possible embodiment of the method, the number of magnetic labeled cells having the same magnetic relaxation time in response to the applied pulsed magnetic field is counted to detect a number of similar cells in a predetermined volume of the supplied flow.

Further aspects will be brought out in the following portions of the specification, wherein the detailed description is for the purpose of fully disclosing embodiments without placing limitations thereon.

### BRIEF DESCRIPTION OF THE DRAWINGS

These and other aspects and advantages will become more apparent and more readily appreciated from the following description of the exemplary embodiments, taken in conjunction with the accompanying drawings of which:

FIG. 1 is a side view cut through a possible exemplary embodiment of a magnetic flow cytometry apparatus;

FIG. 2 is a top view for illustration a possible embodiment of a magnetic flow cytometry apparatus;

FIGS. 3-6 are schematic illustrations of different magnet arrangements of possible embodiments of a magnetic flow cytometry apparatus;

FIG. 7 is a diagram for illustrating a further possible embodiment of a magnetic flow cytometry apparatus;

FIG. 8 is a signal diagram for illustrating a method for detection of labeled cells;

FIG. 9 is a flow chart illustrating a possible embodiment of a method for detection of cells labeled with magnetic nanoparticles.

### DETAILED DESCRIPTION OF EMBODIMENTS

Reference will now be made in detail to the exemplary embodiments which are illustrated in the accompanying drawings, wherein like reference numerals refer to like elements throughout.

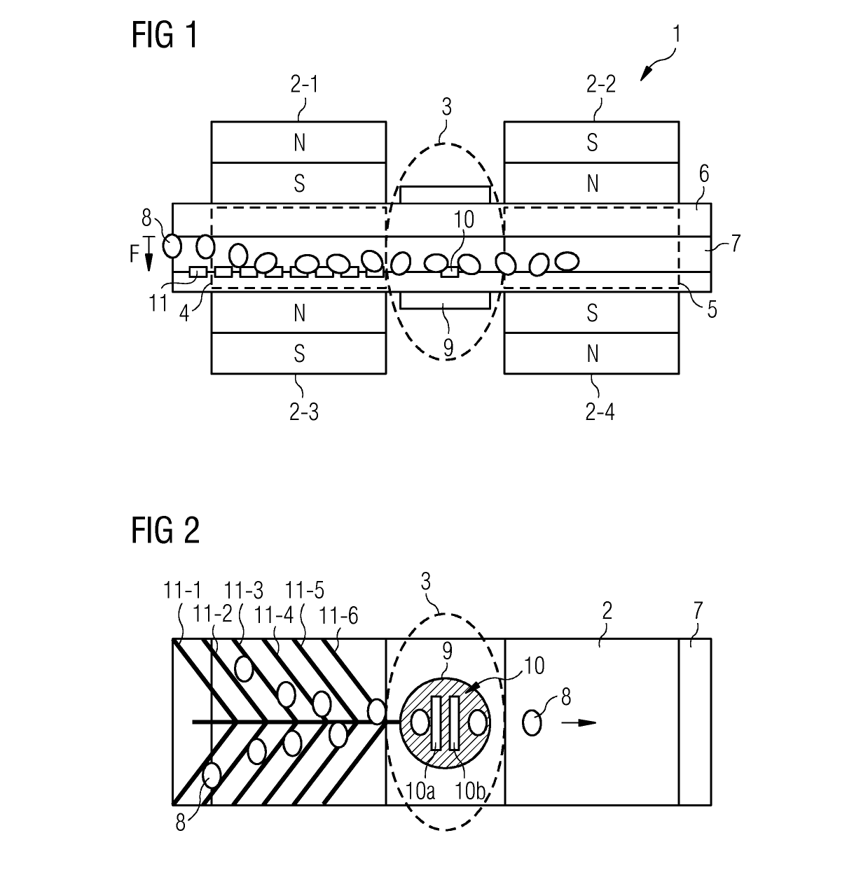

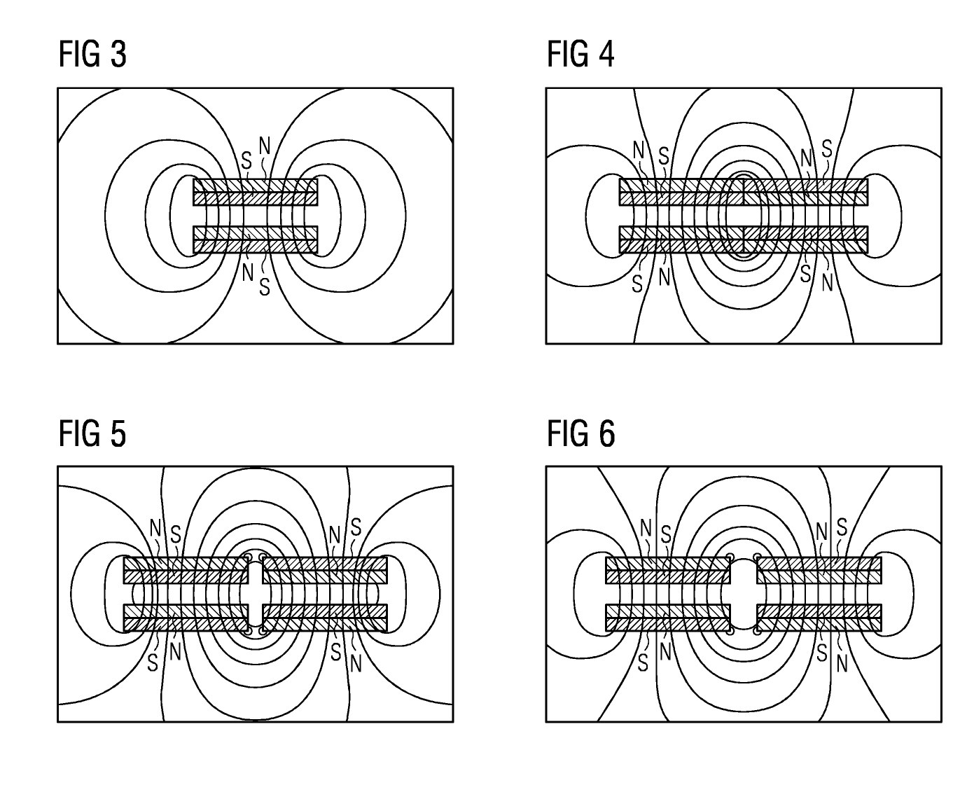

FIG. 1 shows a diagram for illustrating a possible embodiment of a magnetic flow cytometry apparatus 1 for detection of cells labeled with magnetic nanoparticles. The apparatus 1 includes at least one pair of oppositely oriented magnets to provide between the magnets a first magnetic field region with a low magnetic field strength. In the shown exemplary embodiment of FIG. 1 the magnetic flow cytometry apparatus 1 includes four magnets 2-1, 2-2, 2-3, 2-4. A first pair of oppositely oriented magnets 2-1, 2-2 and a second pair of oppositely oriented magnets 2-3, 2-4 is provided in the shown embodiment to generate a first magnetic field region 3 with a low magnetic field strength. The generated magnetic field is also shown for this embodiment in FIG. 6. As can be seen in FIG. 6 in comparison to the other possible configurations of magnets as shown in FIG. 3, 4, 5 the embodiment as shown in FIG. 1 having the magnetic field as shown in FIG. 6 has the advantage that the magnetic field region 3 with a low magnetic field strength is comparatively huge so that magnetic sensors can be easily placed in that region. As shown in FIG. 1 the first magnetic region 3 with a low magnetic field strength is shown as a region with dashed lines. The pairs of oppositely oriented magnets 2-i further provide second magnetic field regions each having a high magnetic field strength. In particular, between the first magnet 2-1 and the third magnet 2-3 a first area 4 with a high magnetic field strength is provided. Similar, in the area between the second magnet 2-2 and the fourth magnet 2-4 also a magnetic field region 5 is provided having a high magnetic field strength. In FIG. 1 two regions 4, 5 are shown having a high magnetic field strength. In a possible embodiment the first magnetic field region 3 includes a low magnetic field strength of less than 1 m Tesla. Further, the two second magnetic field regions 4, 5 each includes a high magnetic field strength of more than 10 m Tesla. The arrangement of the magnets 2-1, 2-2, 2-3, 2-4 is in an embodiment symmetrical as shown in FIG. 1. The magnets 2-i can be formed in a possible embodiment by permanent magnets. In an alternative embodiment the magnets 2-i can also be formed by electrical coils which generate corresponding electromagnetic fields. As can be seen in FIG. 1 the first or zero field region 3 is provided between the magnets 2-1, 2-2, 2-3, 2-4 in the center.

As can be seen in FIG. 1 a capillary tube 6 having at least one micro-fluidic channel 7 is arranged between the magnets 2-i and passes through the magnetic field regions 4, 3, 5 from left to right as shown in FIG. 1. The micro-fluidic channel 7 is provided to supply a stream of fluid including magnetic labeled cells 8. The magnetic labeled cells 8 provided within the flow are input into the magnetic flow cytometry apparatus 1 and are enriched in the magnetic field region 4 between the two magnets 2-1, 2-3 as shown in FIG. 1. The arrangement as shown in FIG. 1 provides a gradient of the magnetic field along the input flow in the magnetic flow cytometry apparatus 1. The gradient of the magnetic field between the first magnet 2-1 and the third magnet 2-3 causes a magnetophoretic force F on the labeled cells.

The magnetophoretic force F on a magnetic bead is given by:

$$F = ∇ ( \frac{1}{2} BH ) V$$

with B=μ₀(1+x)H

for a linear magnetic medium with a volume susceptibility X of about 1 for common magnetic beads, Wherein the vacuum magnetic permeability μ₀=4π·10⁻⁷ H/m and V=(4/3)×πR³ is the volume of the respective bead. For a cell labeled with a thousand magnetic beads or magnetic nanoparticles of 200 nm diameter in a magnetic field gradient of 100 m Tesla over a distance of 1 mm this results in a magnetophoretic force F=30 pN. In comparison, the stoves drag of a fluid on a sphere is: Fₛ=6πηRV, where η is the viscosity of the fluid, e.g. 1 m Pas for water, 2R is the diameter of the cell which can be typically around 3 μm or larger and V is the velocity of the fluid, e.g. around 3 mm/sec. The resulting force Fₛ is about 100 pN, i.e. several times larger than the magnetophoretic force of the magnetic field within the magnetic field region 4. As can be seen in FIG. 1 labeled cells 8 labeled with magnetic nanoparticles are enriched in the magnetic field region 4 having a high magnetic field strength. The magnetophoretic force F causes the labeled cells 8 downward to the lower bottom of the micro-fluidic channel 7 as shown in FIG. 1.

After the enrichment has been performed the labelled cells 8 are supplied to the first magnetic field region 3. In the first magnetic field region 3 having a low magnetic field strength a magnetic field is applied to the enriched magnetic labeled cells 8 to measure a magnetic relaxation of the magnetic labeled cells 8 in response to the applied pulsed magnetic field. In a possible embodiment the applied magnetic field is a pulsed magnetic field. In an alternative embodiment the applied magnetic field is modulated sinusoidally. In a possible embodiment at least one excitation coil 9 is provided close to the first magnetic field region 3 and is adapted to generate the pulsed or sinusoidal magnetic field applied to the enriched magnetic labeled cells 8 flowing at the lower bottom of the micro-fluidic channel 7 as shown in FIG. 1. In a possible embodiment an excitation wire is located close to the first magnetic field region 3 and is adapted to generate the magnetic field applied to the enriched magnetic labeled cells 8. The applied magnetic field is adapted to excite the superparamagnetic nanoparticles attached to the cells. As can be seen in FIG. 1 at least one magnetic sensor 10 is provided within the first magnetic field region 3 and is adapted to measure the magnetic relaxation of the magnetic labeled cells 8 to which the magnetic field is applied. If a pulsed magnetic field is applied the magnetic relaxation is measured by observing a decay time of the magnetic relaxation in the time domain.

If a sinusoidally modulated magnetic field is applied a phase of the magnetization signal can be measured to derive a magnetic relaxation in the frequency domain. It can be more convenient to measure the relaxation in the frequency domain because it is easier to drive an electro magnet harmonically than to pulse it. The pulsed magnetic field and the relaxation measurement are on a time scale typically in the nanosecond/millisecond region which is not blurred by the continuous flow of the labeled cells 8 over the magnetic sensor 10. Preferably, the time constant of the relaxation measurement is at least 10 times faster than the time scale of the cell movement of the labeled cells 8 over the magnetic sensor 10, typically 0,1-10 ms. In a possible embodiment the magnetic sensor 10 is formed by magnetic sensor elements or magnetoresistive sensor elements. The magnetic sensor elements can be GMR, TMR or AMR sensor elements. The magnetic sensor elements of the magnetic sensor 10 can be connected in a measurement bridge circuit such as a wheatstone bridge circuit. The excitation pulse length of a pulse of the pulsed magnetic field applied to the enriched magnetic labeled cells 8 is shorter than the passage time of the magnetic labeled cells 8 when passing the magnetic sensor 10.

As can be seen in FIG. 1 the micro-fluidic channel 7 within the capillary tube 6 is not located in the middle of the capillary tube 6 but is asymmetrically shifted to that side where the magnetic sensor 10 within the magnetic field region 3 is located. The magnetic labeled cells 8 pass the magnetic sensor 10 very closely so that the magnetic sensor 10 can detect a magnetic stray field caused by the magnetic nanoparticles of the labeled cells 8. The low magnetic field within the magnetic field region 3 prohibits that the beads or magnetic nanoparticles of the labelled cells 8 are in magnetic saturation. Accordingly, the magnetic sensor 10 can detect a magnetic relaxation and a magnetic non-linearity, i.e. the variation of the labels magnetization responses to the applied magnetic field generated by the excitation coil 9.

The strong magnetic field region 4 provides for sample enrichment and magnetic guiding of the labeled cells 8 so that they can pass over the magnetic detector 10 closely. The magnetic labeled cells 8 are supplied to the magnetic cytometry apparatus 1 in the micro-fluidic channel 7 in a laminar flow. In an embodiment, the magnetic labeled cells 8 enriched in the high magnetic field region 4 are aligned by ferro-magnetic lines 11 as also shown in FIG. 1. The ferro-magnetic lines 11 can be provided on the inner surface of the micro-fluidic channel 7. In a possible embodiment the labeled cells 8 are aligned to a single file of cells which pass the magnetic sensor 10.

The magnetic field generated by the excitation coil 9 can be applied to the enriched and aligned magnetic labeled cells 8 continuously or discontinuously. In a discontinuous measurement the presence of a labeled cell 8 is measured with the magnetoresistive device and forms a trigger to apply a pulse. For a continuous readout no trigger is applied and any object located over the magnetoresistive device 10 is probed for its relaxation properties. In a possible embodiment an object is identified with a single labeled cell if a flow rate and/or signal amplitude of the magnetoresistive device or magnetic sensor 10 is within a given range of thresholds. In a possible embodiment the magnetic sensor 10 includes only one magnetic sensor element, however, in an embodiment the magnetic sensor 10 includes multiple magnetic sensors or magnetoresistive devices which can be positioned in the magnetic field region 3 and form several relaxation measurements multiple times to reduce the amount of false positive results or to improve the statistics. With laminar flow conditions the enriched and aligned labeled cells 8 remain close to the magnetoresistive devices in the low magnetic field region 3 after the labelled cells 8 leave the high magnetic field region 4. The gradient of the magnetophoretic force F in the region 4 is low enough so that the labeled cells 8 can leave the high magnetic field region 4 to enter the low magnetic field region 3.

FIG. 2 shows a top view on a magnetic sensor 10 having two magnetoresistive sensor elements 10a, 10b, which can be connected in a half bridge. Further, it can be seen in FIG. 2 that the enriched labeled cells 8 are aligned by ferro-magnetic lines 11-1 to 11-6 provided on the inner surface of the microfluidic channel 7 to pass the magnetic sensor elements 10a, 10b in the centre as shown in FIG. 2. The sensor elements 10a, 10b of the magnetic sensor 10 can be formed in a possible embodiment by GMR (Giant Magnetoresistive) sensor elements. It is also possible that the sensor elements 10a, 10b are formed by TMR or AMR sensor elements. FIG. 2 shows the position of the excitation coil 9 provided for generating the magnetic field applied to the labeled cells 8 during their passage through the low magnetic field region 3.

FIGS. 3-6 show possible arrangements of oppositely oriented magnets 2-i for different embodiments of a magnetic flow cytometry apparatus 1. FIG. 6 shows an embodiment having the same arrangement of magnets 2-i as shown in FIG. 1.

The embodiments having the arrangement as shown in FIG. 3 have a strong magnetic uniform field located in the centre and having two low magnetic field regions at the edges. The arrangement shown in FIG. 4 includes an anti-symmetric pair of magnets with a low magnetic field in the centre. As can be seen in FIG. 4 the low magnetic field region in the centre is small in this embodiment.

FIG. 5 shows a similar arrangement as the embodiment of FIG. 4, however in FIG. 5 the magnets to do touch each other. With the embodiments as shown in FIGS. 3-6 a region with uniform magnetic field strength is achieved by using a pair of planar magnets magnetized axially as illustrated in FIG. 3. The uniform magnetic field region is just outside of the magnets 2-i i on the left and right side. The magnetic flow cytometry apparatus 1 for detection of cells labeled with magnetic nanoparticles can in the shown embodiments of FIGS. 3-6 be conveniently placed in a plane between the permanent magnets 2-i. Other embodiments can include different arrangements of the permanent magnets 2-i than those shown in FIGS. 3-6. Having another pair of magnets magnetized in the opposite direction also results in a low magnetic field region in the centre preceded and followed by more extended high magnetic field regions suitable for sample enrichment. The magnets 2-i may not be in direct contact as shown in the embodiments of FIGS. 5, 6. In the embodiments shown in FIGS. 3-6 the magnets 2-i are formed by permanent magnets. In an alternative embodiment the magnets 2-i can also be formed by electrical coils. The gradient of the magnetic field along the flow in the magnetic flow cytometry apparatus 1 determines the magnetophoretic force on the labeled cells 8. The direction of the magnetic field is not of importance for the operation of the magnetic flow cytometry apparatus 1, i.e. north and south poles of the magnets can be exchanged without any problem.

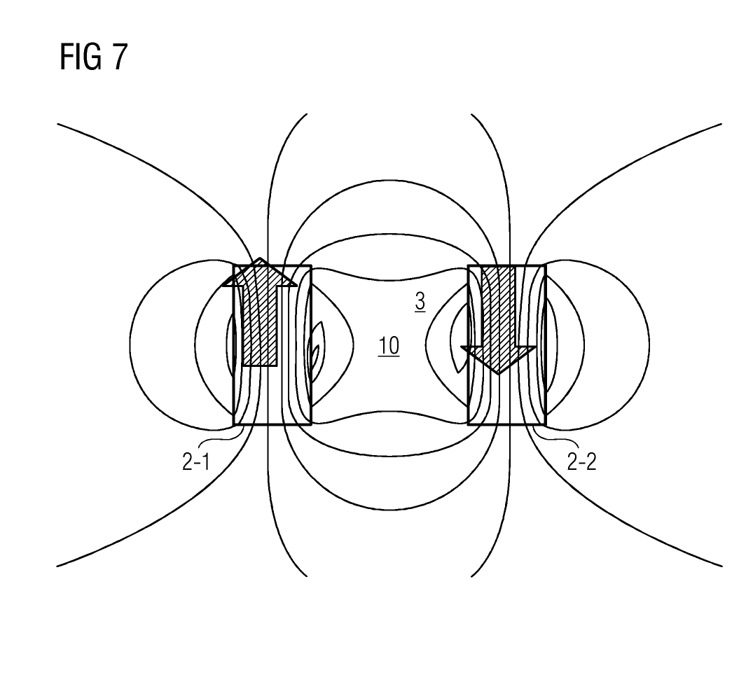

FIG. 7 shows a further possible embodiment of a magnetic flow cytometry apparatus 1. In this example high and low magnetic field regions are formed by two excitation coils forming two magnets 2-1, 2-2 and circling a low field point in the centre. The two coils form magnetic field lines as shown in FIG. 7 and form a pair of oppositely oriented magnets 2-1, 2-2 which provide between the magnets a first magnetic field region 3 with a low magnetic field strength and which provide at poles of the coils second magnetic field regions with a high magnetic field strength.

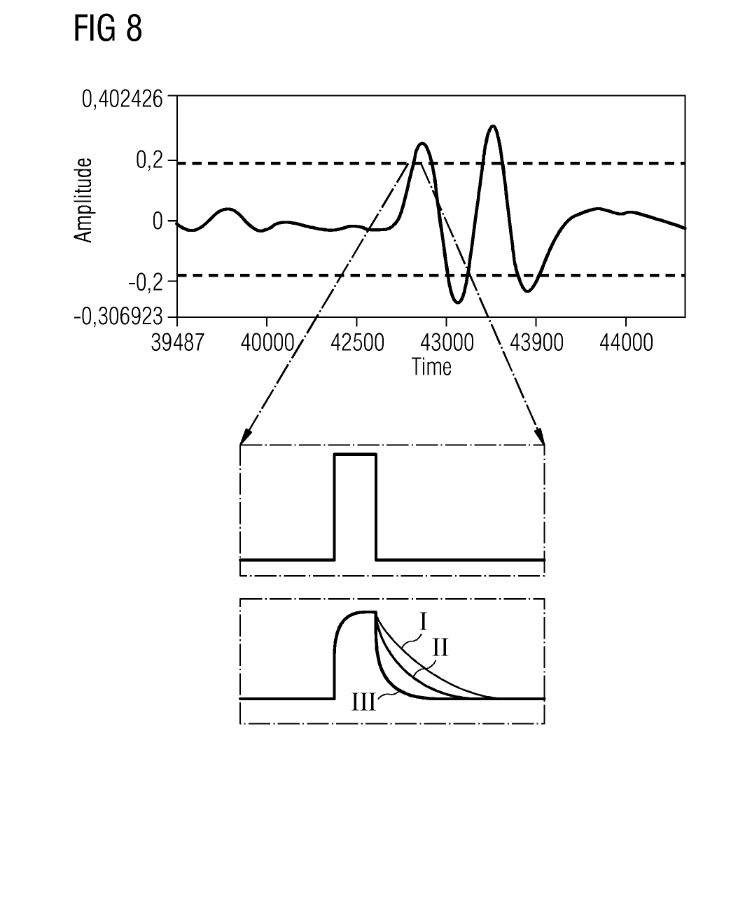

FIG. 8 shows signal diagrams for illustrating the operation of a magnetic flow cytometry apparatus 1 for detection of cells labeled with magnetic nanoparticles. The excitation coil 9 or an excitation wire generates a first magnetic field having excitation pulses formed by rectangular pulses as shown in FIG. 8. The magnetic sensor 10 located close to the low magnetic field region 3 measures a relaxation signal as shown in FIG. 8. In the shown example of FIG. 8 different labels cause different relaxation signals I, II, III allowing to identify magnetic labeled cells 8 having the same measured magnetic relaxation time in response to the applied pulsed magnetic field. In a possible embodiment a cell counter can be provided which counts the number of magnetic labeled cells 8 having the same measured magnetic relaxation time in response to the applied pulsed magnetic field. The magnetic labeled cells 8 having the same magnetic relaxation time in response to the applied pulsed magnetic field can be counted to detect a number of similar cells in a predetermined volume of the supplied flow input into the magnetic flow cytometry apparatus 1. In a possible embodiment the magnetic sensor 10 is formed by magnetic sensor elements 10a, 10b forming a half bridge circuit within a bridge circuit such as a wheatstone bridge circuit. FIG. 8 shows a half bridge signal pattern measured with Giant Magnetoresistive sensor elements. The labeled cells 8 passing through the magnetic sensor elements 10a, 10b cause a half bridge signal pattern as shown in FIG. 8. Thresholds can be used as trigger to perform for example for four repetitive relaxation measurements to improve statistics on relaxation time constants.

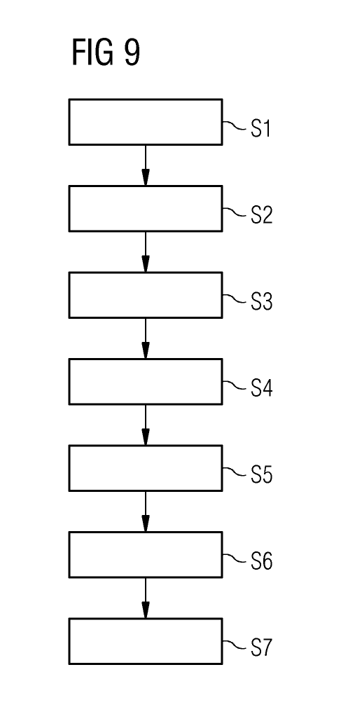

FIG. 9 shows a flow chart for illustrating a possible embodiment of a method for detection of cells labeled with magnetic nanoparticles.

At S1 cells are labeled with magnetic nanoparticles. For example, cells are labeled by adding one or several different markers including magnetic nanoparticles to a fluid container wherein the markers or nanoparticles attach themselves to specific receptors on the surface of the cells. The cells have typically a diameter of 1-20 μm. The cell can have receptors on its surface to which specific other particles such as antibodies can attach themselves. These markers or labels can include magnetic nanoparticles, typically with a size of less than 10 nanometers. These small magnetic particles are superparamagnetic. For example the magnetic nanoparticles can be magnetite particles.

After the cells within the fluid have been labeled (S1) the fluid is applied to magnetic flow cytometry apparatus 1 in a microfluidic channel 7 in a laminar flow (S2).

Next, at S3 the labeled cells 8 of the input flow are enriched in a magnetic field region 4 having a high magnetic field strength.

Then, at S4 the enriched magnetic labeled cells 8 are aligned by ferro-magnetic lines 11 provided on an inner surface of the microfluidic channel 7.

After the labeled cells 8 have reached the low magnetic field region 3 a pulsed or sinusoidal magnetic field is applied at S5 to the enriched magnetic labeled cells 8 in the magnetic field 3 region with the low magnetic field strength.

Next, at S6 the magnetic relaxation of the magnetic labeled cells 8 is measured in response to the applied magnetic field.

Then, at S7 the number of magnetic labeled cells 8 having the same magnetic relaxation in response to the applied magnetic field is counted to detect a number of similar cells in a predetermined volume of the supplied flow.

Accordingly, a magnetic flow cytometry apparatus 1 for detection of cells labeled with magnetic nanoparticles, includes at least one pair of oppositely oriented magnets 2 to provide between the magnets a first magnetic field region 3 with a low magnetic field strength and to provide at poles of the magnets second magnetic field regions 4, 5 with a high magnetic field strength, wherein the magnetic labeled cells 8 provided within a flow input into the magnetic flow cytometry apparatus 1 are enriched in at least one of the second magnetic field regions 4 and supplied to the first magnetic field region 3, where a magnetic field is applied to the enriched magnetic labeled cells 8 to measure the magnetic relaxation of the magnetic labeled cells 8 in response to the applied magnetic field.

It will further be appreciated that the embodiments described above include but are not limited to the following:

1. A magnetic flow cytometry apparatus 1 for detection of cells labeled with magnetic nanoparticles comprising: at least one pair of oppositely oriented magnets 2 to provide between said magnets a first magnetic field region 3 with a low magnetic field strength and to provide at poles of said magnets second magnetic field regions 4, 5 with a high magnetic field strength; wherein the magnetic labeled cells 8 provided within a flow input into said magnetic flow cytometry apparatus 1 are enriched in at least one of the second magnetic field regions 4 and supplied to the first magnetic field region 3; and wherein a magnetic field is applied to the enriched magnetic labeled cells 8 to measure the magnetic relaxation of the magnetic labeled cells 8 in response to the applied magnetic field.

2. The magnetic flow cytometry apparatus according to embodiment 1, wherein the magnetic labeled cells 8 are supplied into said magnetic cytometry apparatus 1 through at least one micro-fluidic channel 7 in a laminar flow.

3. The magnetic flow cytometry apparatus according to embodiment 1, wherein at least one magnetic sensor 10 is provided within the first magnetic field region 3 adapted to measure the magnetic relaxation of the magnetic labeled cells 8.

4. The magnetic flow cytometry apparatus according to embodiment 1, wherein at least one excitation coil 9 or excitation wire is located close to the first magnetic field region 3 and is adapted to generate a pulsed or sinusoidal magnetic field applied to the enriched magnetic labeled cells 8.

5. The magnetic flow cytometry apparatus according to embodiment 1, wherein said at least one pair of oppositely oriented magnets 2 comprises permanent magnets or electrical coils.

6. The magnetic flow cytometry apparatus according to embodiment 1, wherein the first magnetic field region 3 comprises a low magnetic field strength of less than approximately 1 m Tesla.

7. The magnetic flow cytometry apparatus according to embodiment 1, wherein the second magnetic field region 4, 5 comprises a high magnetic field strength of more than approximately 10 m Tesla.

8. The magnetic flow cytometry apparatus according to embodiment 1, wherein the magnetic field is applied to the enriched magnetic labeled cells 8 continuously or discontinuously.

9. The magnetic flow cytometry apparatus according to embodiment 1, wherein a cell counter is provided which counts the number of magnetic labeled cells 8 having the same measured magnetic relaxation time in response to the applied magnetic field.

10. The magnetic flow cytometry apparatus according to embodiment 1, wherein the magnetic labeled cells 8 enriched in the second magnetic field region 4 are aligned by ferro-magnetic lines 11 provided on the inner surface of said micro-fluidic channel 7 to pass the magnetic sensor 10 closely.

11. The magnetic flow cytometry apparatus according to embodiment 10, wherein the excitation pulse length of a pulsed magnetic field applied to said enriched and aligned magnetic labeled cells 8 is shorter than a passage time of the magnetic labeled cells 8 when passing the magnetic sensor 10.

12. The magnetic flow cytometry apparatus according to embodiment 3, wherein the magnetic sensor 10 comprises magnetic sensor elements 10a, 10b connected to a measurement bridge circuit.

13. The magnetic flow cytometry apparatus according to embodiment 12, wherein said magnetic sensor elements 10a, 10b comprise GMR, TMR or AMR sensor elements.

14. A method for detection of cells labeled with magnetic nanoparticles, comprising: enriching S3 magnetic labeled cells 8 of an input flow in a magnetic field region 4 having a high magnetic field strength; applying S5 a pulsed or sinusoidal magnetic field to the enriched magnetic labeled cells 8 in a magnetic field region 4 having a low magnetic field strength; and measuring S6 the magnetic relaxation of the magnetic labeled cells 8 in response to the applied magnetic field.

15. The method according to embodiment 14, wherein the enriched magnetic labeled cells 8 are aligned S4 by ferro-magnetic lines provided on an inner surface of a micro-fluidic channel 7 adapted to supply said input flow of enriched magnetic cells 8 to the magnetic field region 3 having a low magnetic field strength.

16. The method according to embodiment 14, wherein the magnetic field regions 3, 4, 5 are generated by at least one pair of oppositely oriented magnets 2.

17. The method according to embodiment 14, wherein magnetic labeled cells 8 are supplied S2 as an input flow from a fluid container comprising a fluid containing magnetic labeled cells 8.

18. The method according to embodiment 17, wherein cells are labeled S1 by adding one or several different markers each comprising magnetic nanoparticles to said fluid container wherein the markers attach themselves to specific receptors on the surface of the cells.

19. The method according to embodiment 14, wherein the number of magnetic labeled cells 8 having the same magnetic relaxation time in response to the applied pulsed magnetic field is counted S7 to detect a number of similar cells in a predetermined volume of the supplied flow.

The method and apparatus described above can be used for detection of any cells or organic particles. It can be used for example for a quality control in a food production facility producing, for example milk products. Further, the magnetic flow cytometry apparatus 1 can also be used in a medical apparatus for detection of organic cells, for example the number of thrombocytes within a volume. The sample preparation is very easy since the magnetic labels or markers only have to be added to the fluid volume to be inspected. Further preparation is not necessary, in particular no hemolysis must be performed and no separation by centrifugal forces have to be performed. The apparatus and method described above uses a magnetization to distinguish cells being labeled with different superparamagnetic markers having different relaxation or decay times in their magnetization properties.

Although the description above contains many details, these should not be construed as limiting the scope of the invention but as merely providing illustrations of some of the embodiments. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only”one unless explicitly so stated, but rather “one or more.” All structural, chemical, and functional equivalents to the elements of the above-described embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A magnetic flow cytometry apparatus for detection of cells labeled with magnetic nanoparticles, comprising:
at least one pair of oppositely oriented magnets to provide between said magnets a first magnetic field region with a low magnetic field strength and to provide at poles of said magnets second magnetic field regions with a high magnetic field strength;
a supplier of magnetic labeled cells provided within a flow input into said magnetic flow cytometry apparatus, enriched in at least one of the second magnetic field regions and supplied to the first magnetic field region; and
a measurer applying a magnetic field to enriched magnetic labeled cells to measure magnetic relaxation of the magnetic labeled cells in response to the magnetic field applied.

2. The magnetic flow cytometry apparatus according to claim 1, wherein the magnetic labeled cells are supplied into said magnetic cytometry apparatus through at least one micro-fluidic channel in a laminar flow.

3. The magnetic flow cytometry apparatus according to claim 1, further comprising at least one excitation coil or excitation wire located close to the first magnetic field region and adapted to generate a pulsed or sinusoidal magnetic field applied to the enriched magnetic labeled cells.

4. The magnetic flow cytometry apparatus according to claim 1, wherein said at least one pair of oppositely oriented magnets comprises at least one of permanent magnets and electrical coils.

5. The magnetic flow cytometry apparatus according to claim 1, wherein the first magnetic field region comprises a low magnetic field strength of less than approximately 1 m Tesla.

6. The magnetic flow cytometry apparatus according to claim 1, wherein the second magnetic field region comprises a high magnetic field strength of more than approximately 10 m Tesla.

7. The magnetic flow cytometry apparatus according to claim 1, wherein the magnetic field is applied to the enriched magnetic labeled cells continuously or discontinuously.

8. The magnetic flow cytometry apparatus according to claim 1, further comprising a cell counter which counts a number of magnetic labeled cells having a same measured magnetic relaxation time in response to the magnetic field applied.

9. The magnetic flow cytometry apparatus according to claim 1, wherein said measurer includes at least one magnetic sensor provided within the first magnetic field region and adapted to measure the magnetic relaxation of the magnetic labeled cells.

10. The magnetic flow cytometry apparatus according to claim 9, wherein the magnetic labeled cells enriched in the second magnetic field region are aligned by ferro-magnetic lines provided on an inner surface of the micro-fluidic channel to pass said at least one magnetic sensor closely.

11. The magnetic flow cytometry apparatus according to claim 10, wherein an excitation pulse length of a pulsed magnetic field applied to the enriched magnetic labeled cells after alignment is shorter than a passage time of the magnetic labeled cells when passing said at least one magnetic sensor.

12. The magnetic flow cytometry apparatus according to claim 10, wherein said at least one magnetic sensor comprises magnetic sensor elements connected to a measurement bridge circuit.

13. The magnetic flow cytometry apparatus according to claim 12, wherein said magnetic sensor elements comprise GMR, TMR or AMR sensor elements.

14. A method for detection of cells labeled with magnetic nanoparticles, comprising:
enriching magnetic labeled cells of an input flow in a magnetic field region having a high magnetic field strength to obtain enriched magnetic labeled cells;
applying a pulsed or sinusoidal magnetic field to the enriched magnetic labeled cells in a magnetic field region having a low magnetic field strength; and
measuring a magnetic relaxation of the magnetic labeled cells in response to the magnetic field applied.

15. The method according to claim 14, further comprising aligning the enriched magnetic labeled cells by ferro-magnetic lines provided on an inner surface of a micro-fluidic channel adapted to supply the input flow of the enriched magnetic cells to the magnetic field region having a low magnetic field strength.

16. The method according to claim 14, further comprising generating the magnetic field regions by at least one pair of oppositely oriented magnets.

17. The method according to claim 14, further comprising supplying the magnetic labeled cells as the input flow from a fluid container of a fluid containing magnetic labeled cells.

18. The method according to claim 17, further comprising labeling the magnetic labeled cells by adding one or several different markers, each comprising magnetic nanoparticles, to the fluid container to attach the markers to specific receptors on a surface of the magnetic labeled cells.

19. The method according to claim 14, further comprising counting a number of the magnetic labeled cells, having a same magnetic relaxation time in response to the pulsed magnetic field applied, to detect a number of similar cells in a predetermined volume of the input flow.
